(19) United States
(12) Patent Application Publication
(10) Pub. No.: US 2021/0317179 A1
(43) Pub. Date: Oct. 14, 2021

(54) PRECISELY ENGINEERED STEALTHY MESSENGER RNAS AND OTHER POLYNUCLEOTIDES

(71) Applicant: Kernal Biologics, Inc., Cambridge, MA (US)

(72) Inventors: Yusuf Erkul, Quincy, MA (US); Burak Yilmaz, Auburndale, MA (US)

(21) Appl. No.: 17/267,299

(22) PCT Filed: Aug. 8, 2019
(86) PCT No.: PCT/US19/45748

§ 371 Date: Feb. 9, 2021

Related U.S. Application Data
(60) Provisional application No. 62/716,451, filed on Aug. 9, 2018.

(51) Int. Cl. C07K 14/705; A61K 31/7088

(52) U.S. Cl. CPC C07K 14/705; A61K 31/7088

(57) ABSTRACT

Present disclosure is directed to methods of lowering immunogenicity in long polynucleotide sequences by precise sequence engineering of immunogenic motifs in the polynucleotide sequences. This disclosure is further directed to precisely sequence engineered polynucleotides with improved functionality, such as displaying low innate immunogenicity, improved stability or high protein expression. In these polynucleotides, immunogenic sequence motifs are removed while conserving the remainder of the sequence. Compared to overall nucleotide alterations, this targeted engineering approach has unique advantages, including less disruption of the natural or optimized polynucleotide sequence, and hence, preservation of high expressivity while enabling stealthiness vis-à-vis the innate immune receptors.

### INCORPORATION BY REFERENCE OF SEQUENCE LISTING

The Sequence Listing in the ASCII text file, named as Sequence Listing Kernal.txt of 6 KB, created on Aug. 9, 2018, and submitted to the United States Patent and Trademark Office via EFS-Web, is incorporated herein by reference.

### BACKGROUND

The messenger ribonucleic acid (mRNA) field has multiple applications in modern medicine. Of critical importance to the use of mRNA for therapeutic purposes is the reduction of its innate immunogenicity, which otherwise results in a series of undesired effects ranging from cytokine secretion to RNA degradation and stalled translation. Several innate immune receptors have been identified in humans that recognize exogenous mRNAs commonly manufactured via an in vitro transcription (IVT) reaction, which can result in both single-stranded and capped mRNAs, as well byproducts such as double stranded and/or uncapped mRNAs (Sahin et al., 2014, Nat Rev Drug Discov, 13:759-80). The receptors of the innate immune system include sensors of uncapped RNA, double stranded RNAs (dsRNAs), and single stranded RNAs (ssRNA) (Schlee & Hartmann, 2016, Nat Rev Immunol, 16:566-580). Among these receptors, RIG-I binds blunt-ended dsRNAs with 5′ triphosphates (5′PPP) or Cap 0 structure (Schuberth-Wagner et al., 2015, Immunity. 43:41-52), whereas IFIT1 binds ssRNAs with 5′ triphosphates (5′PPP) or Cap 0 structure (Abbas et al., 2013, Nature. 494:60-64; Abbas et al., 2017, PNAS, 114:E2106-E2115). These uncapped RNA sensors can be evaded by efficient capping to obtain Cap I structure, and/or by phosphatase treatment of IVT mRNA (Warren et al., 2010, Cell Stem Cell. 7:618-30; Ramanathan et al., 2016, Nucleic Acids Res. 44:7511-7526). Receptors that sense dsRNAs include TLR3, MDA5, PKR and OAS1 (Schlee & Hartmann, 2016, Nat Rev Immunol, 16:566-580), which can be evaded by purification of mRNA to remove the double stranded RNA products of the IVT reaction (Karikó et al., 2011, Nucleic Acids Res. 39:e142; Person et al. 2014, USPTO Patent App No: US 2014/0328825 A1).

Innate immune receptors that bind ssRNAs (single stranded ORNs and individual strands of siRNA duplexes) include TLR7 and TLR8, which are highly homologous (Wang et al., 2006, J Biol Chem, 281:37427-37434; Matsushima et al., 2007, BMC Genomics, 10.1186/1471-2164-8-124; Wei et al. 2009, Protein Sci., 18:1684-1691). Double stranded RNAs including siRNAs can also be recognized by TLR7 and TLR8 after separation of the two strands of double stranded RNA into single stranded RNAs within the endosome (Goodchild et al., 2009, BMC Immunology, 10:40). Upon stimulation of these receptors, intracellular NE-KB and IRF-3 signaling pathways are activated and this in turn results in the secretion of IFN-alpha (TLR7) and TNF-alpha and IL-12p40 (TLR8) (Gorden et al. 2005; J Immunol., 174:1259-1268; Forsbach et al., 2008, J Immunol., 180:3729-3738). Chrystal structures of these proteins were recently solved (Tanji et al., 2015, Nat Struct Mol Biol. 22:109-115; Zhang et al., 2016, Immunity. 45:737-748) and their ligand binding sites were identified (Wei et al., 2009, Wei et al. 2009, Protein Sci., 18:1684-1691; Ohto et al., 2014, Microbes Infect. 16:273-282). These studies revealed two separate ligand binding domains: one binding a single nucleoside (guanosine for TLR7 and uridine for TLR8) and another binding a short oligoribonucleotide (ORN). Ligand binding at both domains is required to dimerize and activate these receptors. Structural biology studies are in alignment with previous work on TLR7/8 ligands, which have consistently shown U- and GU-rich ORN sequences to be activators of TLR7/8 (Judge et al. 2005, Nat Biotechnol, 23, 457-462; Heil et al., 2004, Science, (80)303:1526-1529; Hornung et al., 2005, Nat Med., 10.1038/nm1191). Several groups identified specific ssRNA sequences that had high stimulatory activity for TLR7 and/or TLR8 (Diebold et al., 2006, Eur J Immunol. 10.1002/eji.200636617; Forsbach et al., 2008, J Immunol., 180:3729-3738; Jurk et al., 2011, Nucleic Acid Ther. 21:201-214, Green et al., 2012, J Biol Chem. 287:39789-39799). Jurk et al. (2011) tested various derivatives of these ssRNAs and identified ssRNA sequence motifs for TLR7/8 binding. They noted UCW motif (where W is U or A) for human TLR7 (based on IFN-α secretion) and KNUNDK motif (where N is any nucleotide, K is G or U, and D is any nucleotide but C) for human TLR8 stimulation (based on IL12p40 secretion).

Purified and capped IVT mRNA can evade RIG-I, IFIT, PKR, MDA5, OAS, and TLR3 but is recognized by TLR7 and TLR8 in human cells. This recognition can be avoided either by incorporation of non-canonical nucleotides, such as pseudouridine, N1-methyl-pseudouridine, methoxy-uridine, and 2-thiouridine into mRNA (Kariko, 2005, Immunity. 23:165-75; Kariko, 2008, Mol Ther. 16:1833-40; Kormann et al., 2011, Nat Biotechnol. 29:154-157; Andries et al., 2015, J Control Release. 217:337-344) or by unrefined/crude engineering of mRNA sequence via altering the overall nucleotide content of mRNA. The latter approach can be done via increasing GC content (Thess et al., 2015, Mol Ther. 23:1456-64; Schlake and Thess, 2015) or increasing A or decreasing U or GU content of mRNAs (Kariko & Sahin, 2017, WIPO Patent App No: WO 2017/036889 A1). For the coding region, this sequence engineering is done by mainly changing the 3^{rd} nucleotides of the codons on mRNA. Due to the redundancy in genetic code, sequence engineering does not alter the amino acid sequence of the encoded protein. This method is similar to codon optimization, a technique commonly used in molecular and synthetic biology to improve the protein expression yields of transgenes (Quax et al., 2015, Mol Cell. 59:149-161) However, in the case of IVT mRNA sequence engineering, the primary goal is to render IVT mRNAs stealthy or invisible to RNA sensors in the body.

Chemical modifications such as pseudouridine reduce, but do not completely ablate innate immunogenicity, particularly upon repeated transfections (Liang et al., 2017, Mol Ther. 25(12):2635-2647). In addition, there are possible therapeutic uses of mRNA where stimulation of some, but not other RNA sensors may be desirable. For instance, mRNAs with only TLR7 binding activity may be desirable in some immuno-oncology applications where IFN-alpha secretion can induce or boost antitumor immunity. Chemical modifications do not allow for evasion of some sensors and stimulation of others.

The GC content of the coding regions within humans genome is 52% (Merchant et al., 2007, Science. 318(5848):245-50) and less than 1% of its nucleotides are non-canonical (Li et al, 2015, Nat Chem Biol, 11(8):592-7). As mRNA chemistry or sequence is modified further away more from natural (cellular) human mRNA (to reduce the innate immunogenicity of IVT mRNA), the risk of having unintended consequences increases. Both chemical modifications and sequence engineering via overall nucleotide content alteration approach are unrefined/crude methods which can be disruptive and can have complications; such as reduced translation (for 5-methyl-cytidine, 6-methyladenosine, and 2-thio-uridine modifications) (Kariko et al., 2015, Mol Ther, 16(11):1833-40) or cryptic peptide formation (Mauro & Chappell, 2014, Trends Mol Med. 2014 November; 20(11):604-13; Mauro et al., 2018, BioDrugs, 32:69-81). Furthermore, within the human mRNA “epitranscriptome,” chemically modified nucleosides such as m6A and pseudouridine are not uniformly distributed (Carlile et al., 2014, Nature. 515:143-6; Dominissini et al., 2016, Nature. 530:441-446). For instance, uridines located in mammalian stop codons do not contain pseudouridylation motifs (Schwartz et al, 2014, Cell. 159:148-162) and pseudouridine incorporation into IVT mRNA was shown to cause stop codon readthrough (Karijolich & Yu, 2011, Nature. 474:395-398; Fernandez et al, 2013, Nature. 500:107-110). Furthermore, modified nucleotides can reduce the fidelity of RNA transcription enzyme (T7 RNA polymerase) as well as the translation machinery and can also alter post-translational modification of proteins, Modified nucleotides also render mRNA resistant to RNases in humans, and RNA accumulation in serum can cause hypercoagulable states. In addition to these biological risks, the use of non-canonical nucleotides can also lead to increased manufacturing costs (Hadas et al., 2017, Wiley Interdiscip Rev Syst Biol Med. 9:e1367).

### SUMMARY OF THE DISCLOSURE

This invention provides polynucleotides (e.g., messenger RNAs) that are sequence engineered to remove immunogenic sequence motifs implicated in binding to human TLR8.

In one embodiment, the present invention provides a method of precise sequence engineering for polynucleotides (e.g., mRNA) where only the immunogenic motifs are removed while the rest of the sequence remains intact.

In one embodiment, the present invention provides a method of removing an immunogenic RNA sequence motif, KNUNDK, from a polynucleotide (e.g., mRNA), which significantly reduces innate immunogenicity via human TLR8.

In one embodiment, the present invention provides, a messenger RNA encoding GFP where one or more immunogenic sequences that match the KNUNDK sequence motif within the coding region of the mRNA are removed via codon engineering of the DNA template for the sequence and is transfected to HEK cells to show reduced immunogenicity via human TLR8 and high protein expression.

One aspect of the present invention is a method comprising repeatedly contacting a human embryonic kidney cell line (HEK293-TLR8 SEAP) with a KNUNDK sequence motif removed mRNA to enable high levels of protein expression while reducing the innate immunogenicity of the mRNA.

One aspect of the present invention is a method comprising contacting a human primary monocyte derived dendritic cells (MDDC) with a KNUNDK motif removed mRNA to enable high levels of protein expression while reducing the innate TLR8 immunogenicity of the mRNA.

Another aspect of the present invention is a novel, precise stealthy mRNA engineering method that prevents human TLR8 activation by the mRNA, while allowing for activation of other RNA sensors, such as human TLR7 and human RIG-I.

Precise mRNA engineering methods disclosed herein: via motif removal, spare the non-immunogenic sequences within the mRNA while removing the immunogenic sequences. This minimally invasive approach allows mRNA to retain high levels of translation activity while reducing its immunogenicity. Unlike the crude sequence engineering approach (such as high GC, low GU, or low-U based mRNA engineering), this approach does not disrupt efficient translation, therefore it does not require testing of many versions of sequence engineered mRNA to preserve or attain high levels of protein expression. Because this approach does not involve the use of non-canonical nucleotides, issues such as decreased translation efficiency, post-translational alterations or stop codon readthrough are not expected. Finally, precise engineering can also reduce the manufacturing costs of mRNA therapeutics.

### BRIEF DESCRIPTION OF THE DRAWINGS

FIGS. 1A-1B. A. Sequence engineered eGFP mRNA designs. Native (“Wild Type”) mRNA sequence was altered within coding region to remove TLR8 motifs (“Low motif”), decrease overall G and U content (“Crude”), or both remove motifs and reduce G and Us (“Low motif+Crude”). FIG. 1. B. Summary of nucleotide and motif changes. For each mRNA design approach, the final number of TLR8 motifs present and the total number of altered nucleotides are shown in the table. Precise (low motif) approach efficiently removes TLR8 binding sites while minimizing the number of nucleotides altered. (UTR: untranslated region).

FIG. 2. Innate immunogenicity of engineered eGFP mRNAs transfected via Lipofectamine to human cells overexpressing TLR8. Wild type (WT) and sequence engineered mRNAs were purified via HPLC and transfected via Lipofectamine 2000 (Life Technologies) to HEK293 cells that overexpress TLR8 and carry a reporter plasmid results in the secretion of secreted embryonic alkaline phosphatase (SEAP) upon TLR8 stimulation (via IFN-B promoter fused to NF-KB and AP-1 binding sites). Secreted SEAP activity was measured 48 hours after mRNA transfection. Low motif, crude (low GU), and low motif+crude mRNAs showed significantly reduced TLR8 stimulation compared to the wild type (WT) mRNA (p<0.05 for all 3 comparisons). Transfections were performed in quantiplicates and data is depicted as mean+1-standard deviation (SD).

FIG. 3. Innate immunogenicity of engineered eGFP mRNAs transfected via Trans-IT in human cells overexpressing TLR8. Wild type (WT) and sequence engineered mRNAs were purified via HPLC and transfected via TransIT-mRNA reagent (Mirus Bio) to HEK293-null cells (without TLR8 expression) and HEK293-TLR8 cells that overexpress TLR8. Both cell lines carry a reporter plasmid that results in the secretion of alkaline phosphatase (SEAP) upon TLR8 stimulation (via IFN-B promoter fused to NF-KB and AP-1 binding sites). Secreted AP activity was measured 24 hours after mRNA transfection and normalized to cell number quantitated by pre-experimental SEAP levels. Chemically modified (“chem. mod.”) mRNA control contained 100% pseudo-U and 100% 5mC. (AP activity in HEK-Null cells was measured to determine background immune signal driven via basal TLR3 expression). Similar to chemically modified mRNA, low motif mRNA showed significantly lower TLR8 stimulation than wild type (WT) and Low GU (“Crude”) mRNAs. Transfections were performed in quantiplicates and data is depicted as mean+/−SD.

FIGS. 4A-4B. A. Protein expression driven by engineered eGFP mRNAs transfected via Lipofectamine 2000 to human cells overexpressing TLR8. Wild type (WT) and sequence engineered mRNAs were purified via HPLC and transfected via Lipofectamine 2000 to HEK293 cells that overexpress TLR8. Image of eGFP expressing cells was obtained via Envision plate reader 2 days after transfection. B. Quantification of eGFP expression in human cells overexpressing TLR8 shown in FIG. 4. A. Precisely engineered mRNA (“Low motif”) showed significantly higher protein expression than Low GU (“Crude”) and chemically modified (“Chem. mod.”) mRNAs. Transfections were performed in quantiplicates and data is depicted as mean SD.

FIGS. 5A-5D. Protein expression driven by engineered eGFP mRNAs transfected to human monocyte-derived dendritic cells (MDDCs). Wild type (WT) and sequence engineered mRNAs were purified via HPLC and transfected via Lipofectamine 2000 to MDDCs. eGFP expression was quantified on day 4. Following a single transfection in MDDCs, low motif mRNA (C) resulted in significantly higher protein expression than crude mRNA (B) and similar expression to WT mRNA (A). (D) Results of experiments performed in triplicates. Data depicted as mean+/−SD.

FIG. 6. Protein expression driven by engineered eGFP mRNAs repeatedly transfected via Lipofectamine 2000 to human cells overexpressing TLR8, Wild type (WT) and sequence engineered mRNAs were either collected via a spin column (“WT—unpurified” and “Low Motif—Unpurified”) or purified via HPLC (“WT—HPLC” and “Low Motif—HPLC”). They then were transfected consecutively on days 2, 3 and 4 via Lipofectamine 2000 to HEK2Y3 cells overexpressing TLR8 (seeded on day 0), eGFP expression was quantified on day 4, 7, and 11. In repeated transfection setting, low motif purified mRNA showed significantly higher protein expression than WT purified mRNA. Transfections were performed in quantiplicates and data is depicted as mean+/−SD.

### DETAILED DESCRIPTION

### Definitions

As used herein, the term “about” refers to a variation within approximately ±10% from a given value.

The term “cloning site” refers to a nucleotide sequence, typically present in an expression vector, that includes one or more restriction enzyme recognition sequences useful for cloning a DNA fragment(s) into the expression vector. Where a nucleotide sequence contains multiple restriction enzyme recognition sequences, the nucleotide sequence is also referred to as a “multiple cloning site” or “polylinker.”

The term “expression vector” refers to a nucleic acid that includes sequences that effect the expression of a desirable molecule, e.g., a promoter, a coding region and a transcriptional termination sequence. An expression vector can be an integrative vector (i.e., a vector that can integrate into the host genome), or a vector that does not integrate but self-replicates, in which case, the vector includes ““an origin of replication which permits the entire vector to be reproduced once it is within the host cell.

The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

The phrase “immunogenic motif” is used herein to include references to any RNA sequence that is implicated in binding of the RNA to innate immune receptors such as TLR7 or TLR8 located within cells and causes the activation of intracellular cell signaling pathways resulting in altered gene expression and/or release of cytokines from cells.

The term “plasmid” includes both naturally occurring plasmids in bacteria, and artificially constructed circular DNA fragments.

As used herein, the term “polynucleotide” refers to any RNA or DNA sequence that is longer than 13 nucleotides. The term polynucleotide includes nucleic acids of natural or synthetic origin, with natural or synthetic (chemically modified) phosphate backbones, sugars, and ribose sugars.

As used herein, the term “messenger RNA” and “mRNA” refer to any RNA sequence that is capable of encoding polypeptides or proteins in cells or in cell-free protein translation systems.

As used herein, the term “in vitro transcription” refers to an enzymatic reaction for manufacturing mRNA from a DNA template, which can be plasmid based or PCR product based. In the former case, the plasmid DNA linearized with restriction enzymes and the IVT template region between restriction sites is purified to obtain higher quality DNA template. In the latter case, primers complementary to the terminal regions or flanking regions are designed to amplify and then purify the template DNA from the plasmid. When one of these PCR primers includes a poly-T sequence it can also enable incorporation of a poly-A tail into the mRNA sequence during transcription. In vitro transcription (IVT) reactions commonly use T7, T3, or SP6 RNA polymerase enzymes with canonical or chemically modified nucleotide substrates.

As used herein, the term “coding region” refers to the part of messenger RNA, generally located in between 5′ and 3′ untranslated regions and is actively translated into a protein by ribosomes.

As used herein, the term “5′UTR” refers to the part of messenger RNA that is located on the 5′ terminal end of the mRNA and is generally involved with binding to the ribosome and enhancing the expression of the mRNA coding region.

As used herein, the term “3′UTR” refers to the part of messenger RNA that is located on the 3′ terminal end of the mRNA and is generally involved with enhancing the expression and half-life of the mRNA.

As used herein, the term “sequence engineering” refers to any changes made on the nucleotide sequence of polynucleotides for specific reasons. Such changes can result in reduced immunogenicity, enhanced expression, and/or enhanced half-life. They can be made throughout the RNA sequence or within a specific section of RNA sequence. For messenger RNA, sequence engineering may involve altering coding sequence, 5′UTRs, and/or 3′UTR regions.

As used herein, the term “precise sequence engineering” refers to changes made in an oligonucleotide sequence to reduce immunogenicity of the oligonucleotide by removal of immunogenic motifs while avoiding unnecessary alterations in the rest of the oligonucleotide sequence. In some embodiments, “precise sequence engineering” involves removing at least 1, 2, 3, 4, 5 immunogenic motifs, or all the immunogenic motifs in a polynucleotide. In some embodiments, “precise sequence engineering” involves removal of at least 10%, at least 20%, at least 30%, at least 40%, at least 50%, at least 60%, at least 70%, at least 80%, at least 90%, at least 99% or 100% of the immunogenic motifs found in a polynucleotide sequence.

The phrase “removal of an immunogenic motif” refers to modification of an immunogenic motif in a polynucleotide by changing a single nucleotide in an immunogenic motif, or multiple nucleotides in an immunogenic motif (e.g., 2, 3, 4, 5, 6, or all the nucleotides of a given immunogenic motif) such that the motif no longer exists (i.e. the immunogenic motif is “destroyed.”). The term “change” as used herein, encompasses modifications to a nucleotide or multiple nucleotides including, but not limited to, nucleotide substitution, deletion, insertion, and chemical modification. For example, the “KNUNDK” immunogenic motif encompasses 192 possible nucleotide sequences as shown in Table 1. Any mutation, change or substitution of one or more nucleotides that results in a sequence that does not conform to the “KNUNDK” motif (i.e., that falls outside the listed 192 sequences listed in Table 1) would “destroy” or “remove” the motif. For instance, if the immunogenic motif in a starting polynucleotide is “GAUAAG” and it is mutated to “GAAAAG” the KNUNDK motif is said to be “removed”.

In some embodiments, the oligonucleotide is an RNA. In a specific embodiment, the RNA is a messenger RNA (mRNA). Within the coding region of an mRNA, precise sequence engineering takes advantages of the redundancy of genetic code and replaces each target codon with an alternative codon that encodes for the same amino acid as the native codon, thereby preserving the final sequence of the encoded protein. In other words, if the at least one immunogenic motif is in the amino acid-encoding part of an mRNA (i.e., in the “open reading frame” or “ORF”), the change (e.g., a change of at least 1, 2, 3, 4, 5, or all nucleotides) is done without changing the amino acid sequence encoded by the mRNA.

As used herein, the term “codon optimization” refers to sequence engineering performed for the purposes of increasing polypeptide or protein expression levels. Methods of measuring the amount or levels of polypeptides and proteins are well known in the art.

As used herein, the term “Low GU mRNA” refers to sequence engineered mRNA that has reduced guanine (G) and uracil (U) content compared to that of the wild type version of the same mRNA.

As used herein, the term “Low U mRNA” refers to sequence engineered mRNA that has reduced U content compared to that of the wild type version of the same mRNA.

As used herein, the term “High GC mRNA” refers to sequence engineered mRNA that has elevated G and C content compared to that of the wild type version of the same mRNA.

As used herein, the term “enzymatic capping” refers to the addition of a 7-methyl Guanosine-based cap structure, such as Cap 0, Cap I, Cap II, by an enzyme, typically Vaccinia capping system, which adds 7-methyl-Guanosine cap (Cap 0) with a 5′-5′ phosphodiester bond, in combination with a 2-O-methyltransferase, which 2-O-methylates the first nucleotide at the 5′end of the mRNA resulting in Cap I structure, which are added following the transcription reaction, to enhance better translation of mRNA. Methods of enzymatic capping are well known in the art.

As used herein, the term “co-transcriptional capping” refers to the addition of a 7-methyl Guanosine cap or a cap analogue, such as ARCA or CleanCap by inclusion of such cap analogues into the mRNA transcription reaction, to enhance better translation of mRNA. Methods of co-transcriptional capping are well known in the art.

As used herein, the term “chemical modification” refers to the chemical alterations made to the nitrogenous bases of mRNA. Such alterations are commonly performed by inclusion of non-canonical (chemically modified) nucleotide analogues as substrates for T7 RNA polymerase in the mRNA transcription reaction. These chemical modifications include, but are not limited to pseudouridine (ψ), 5-methylcytidine (m5C), N1-methyl-pseudouridine (N1mψ), 5-methoxyuridine (5moU), N6-methyladenosine (m6A), 5-methyluridine (m5U), or 2-thiouridine (s2U).

As used herein, the term “partial chemical modification” refers to the chemical modification of some but not all of particular nucleotides, typically uridines or cytidines, within mRNA. For instance, 2-thiouridine (s2U) can be used at approximately 25% rate by partially including it as an IVT substrate at a molar rate of 1 to 3, where for every three canonical uridines, one 2-thiouridine is incorporated into the mRNA.

As used herein, the term “encapsulation” refers to packaging of mRNA within solid, lamellar or vesicle-like, lipid- or polymer-based nanoparticles.

As used herein, the term “delivery vehicle” refers to any natural or synthetic material that can be used for the encapsulation of mRNA and enables effective stabilization, transport, and delivery of mRNA payload into the target cells or tissues.

The phrase “research-use composition” refers to any research material used in the laboratory for the purposes of increasing scientific knowledge and is not intended for clinical or veterinary use. The phrase “veterinary composition” refers to any material that is used in animals to improve the health and wellbeing of animals.

### General Description

The present disclosure is directed to methods of lowering immunogenicity in polynucleotide sequences by precise sequence engineering to remove immunogenic motifs in the polynucleotide sequences. The present disclosure is also directed to compositions of engineered polynucleotides where one or more, or all of the immunogenic sequence motifs in such polynucleotides are removed.

In view of limitations of existing mRNA modification and engineering methods, there continues to be a need for a novel mRNA engineering approach that only alters sequences of relevance vis-à-vis the innate immune sensors.

Currently available mRNA chemical modification and sequence engineering approaches that allow for a reduction of innate immunogenicity of mRNA are too crude and alter or modify all the sequences homogenously.

TLR7 and TLR8 detect ssRNA species including mRNA based on certain U containing sequences or sequence motifs. Targeted removal of these immunogenic motifs can allow for a more precise sequence engineering approach. Due to the redundancy in genetic code (where 3^{rd} positions of nearly all codons have alternative nucleotides that encode the same amino acid residue in nascent polypeptide chain), mRNA sequence can be altered to specifically remove sequence motifs, while the encoded protein sequence remains the same.

Compared to crude engineering approaches such as high GC mRNA (where the mRNA sequence is artificially changed to increase overall G and C content) or low GU mRNA (where the mRNA sequence is artificially changed to decrease overall G and U content), this precise approach (low motif approach) is minimally invasive, i.e. it does not alter any sequences that are not implicated in TLR7/8 binding. As a result, this novel approach maintains most of the structural and functional features of said mRNA. Among many advantages, this approach allows for robust translation efficiency. For the first time, present invention shows that precise mRNA engineering is both feasible and advantageous.

Accordingly, in one embodiment, the motifs described herein may be removed from other messenger RNAs used for expressing proteins for research purposes as well as veterinary and clinical applications such as vaccination or therapeutic gene replacement. Said mRNAs can encode one or more of a variety of oligopeptides, polypeptides or proteins, including but not limited to gene editing enzymes (e.g. Cas9, ZFN, and TALEN), induced pluripotent stem cell (IPSC) reprogramming factors (Oct4, Sox2, Klf4, and c-Myc, Nanog, Lin28, Glis1), trans-differentiation factors, metabolic enzymes (e.g. Surfactant protein B, Uridine 5′-diphospho-glucuronosyltransferase, Methylmalonyl CoA mutase, Ornithine transcarbamylase), cell membrane proteins (e.g. CFTR, OX40L, TLR4, CD40L, CD70, B-cell receptor subunits, T-cell receptor subunits, chimeric antigen receptors), hormones and cytokines (EPO, VEGF, IL12, IL36gamma), pro-apoptotic, necrotic and necroptotic proteins, viral antigens (e.g. HIV gp120 and gp41 antigens, influenza HA and NA antigens), bacterial antigens and toxins, cancer antigens and neo-antigens, prophylactic or therapeutic antibodies and antibody fragments.

In another embodiment, mRNA to be sequence engineered can encode more than one protein, either as chimeric constructs (yielding fusion proteins) or as separate polypeptides encoded by distinct coding regions that are interspersed with an IRES region or a sequence coding for a self-cleaving peptide.

In some embodiments, the present invention utilizes KNUNDK as a human TLR8 and mouse TLR7 motif and removes sequences that match the KNUNDK motif, where N is any nucleotide, K is either Guanosine (G) or Uridine (U), and D is any nucleotide but Cytidine (C). The 6-mer sequences comprising KNUNDK motif are provided in Table 1.

In other embodiments, precise sequence engineering via motif removal can be based on other TLR7 and TLR8 sequence motifs, including but not limited to UCW, UNU, UWN, USU, KWUNDK, KNUWDK, UNUNDK, KNUNUK (Forsbach et al. 2008; Jurk et al. 2011; Green et al. 2012) and combinations thereof, where W is Adenosine (A) or U, and S is G or C.

TABLE 1
List of sequences that match the KNUNDK motif

| # | SEQ | # | SEQ | # | SEQ | # | SEQ |
|---|---|---|---|---|---|---|---|
| 1 | GAUAAG | 49 | GGUAAG | 97 | UAUAAG | 145 | UGUAAG |
| 2 | GAUAAU | 50 | GGUAAU | 98 | UAUAAU | 146 | UGUAAU |
| 3 | GAUAUG | 51 | GGUAUG | 99 | UAUAUG | 147 | UGUAUG |
| 4 | GAUAUU | 52 | GGUAUU | 100 | UAUAUU | 148 | UGUAUU |
| 5 | GAUAGG | 53 | GGUAGG | 101 | UAUAGG | 149 | UGUAGG |
| 6 | GAUAGU | 54 | GGUAGU | 102 | UAUAGU | 150 | UGUAGU |
| 7 | GAUUAG | 55 | GGUUAG | 103 | UAUUAG | 151 | UGUUAG |
| 8 | GAUUAU | 56 | GGUUAU | 104 | UAUUAU | 152 | UGUUAU |
| 9 | GAUUUG | 57 | GGUUUG | 105 | UAUUUG | 153 | UGUUUG |
| 10 | GAUUUU | 58 | GGUUUU | 106 | UAUUUU | 154 | UGUUUU |
| 11 | GAUUGG | 59 | GGUUGG | 107 | UAUUGG | 155 | UGUUGG |
| 12 | GAUGGU | 60 | GGUGGU | 108 | UAUGGU | 156 | UGUGGU |
| 13 | GAUGAG | 61 | GGUGAG | 109 | UAUGAG | 157 | UGUGAG |
| 14 | GAUGAU | 62 | GGUGAU | 110 | UAUGAU | 158 | UGUGAU |
| 15 | GAUGUG | 63 | GGUGUG | 111 | UAUGUG | 159 | UGUGUG |
| 16 | GAUGUU | 64 | GGUGUU | 112 | UAUGUU | 160 | UGUGUU |
| 17 | GAUGGG | 65 | GGUGGG | 113 | UAUGGG | 161 | UGUGGG |
| 18 | GAUGGU | 66 | GGUGGU | 114 | UAUGGU | 162 | UGUGGU |
| 19 | GAUCAG | 67 | GGUCAG | 115 | UAUCAG | 163 | UGUCAG |
| 20 | GAUCAU | 68 | GGUCAU | 116 | UAUCAU | 164 | UGUCAU |
| 21 | GAUCUG | 69 | GGUCUG | 117 | UAUCUG | 165 | UGUCUG |
| 22 | GAUCUU | 70 | GGUCUU | 118 | UAUCUU | 166 | UGUCUU |
| 23 | GAUCGG | 71 | GGUCGG | 119 | UAUCGG | 167 | UGUCGG |
| 24 | GAUCGU | 72 | GGUCGU | 120 | UAUCGU | 168 | UGUCGU |
| 25 | GUUAAG | 73 | GAUAAG | 121 | UUUAAG | 169 | UAUAAG |
| 26 | GUUAAU | 74 | GAUAAU | 122 | UUUAAU | 170 | UAUAAU |
| 27 | GUUAUG | 75 | GAUAUG | 123 | UUUAUG | 171 | UAUAUG |
| 28 | GUUAUU | 76 | GAUAUU | 124 | UUUAUU | 172 | UAUAUU |
| 29 | GUUAGG | 77 | GAUAGG | 125 | UUUAGG | 173 | UAUAGG |
| 30 | GUUAGU | 78 | GAUAGU | 126 | UUUAGU | 174 | UAUAGU |
| 31 | GUUUAG | 79 | GAUUAG | 127 | UUUUAG | 175 | UAUUAG |
| 32 | GUUUAU | 80 | GAUUAU | 128 | UUUUAU | 176 | UAUUAU |
| 33 | GUUUUG | 81 | GAUUUG | 129 | UUUUUG | 177 | UAUUUG |
| 34 | GUUUUU | 82 | GAUUUU | 130 | UUUUUU | 178 | UAUUUU |
| 35 | GUUUGG | 83 | GAUUGG | 131 | UUUUGG | 179 | UAUUGG |
| 36 | GUUGGU | 84 | GAUGGU | 132 | UUUGGU | 180 | UAUGGU |
| 37 | GUUGAG | 85 | GAUGAG | 133 | UUUGAG | 181 | UAUGAG |
| 38 | GUUGAU | 86 | GAUGAU | 134 | UUUGAU | 182 | UAUGAU |
| 39 | GUUGUG | 87 | GAUGUG | 135 | UUUGUG | 183 | UAUGUG |
| 40 | GUUGUU | 88 | GAUGUU | 136 | UUUGUU | 184 | UAUGUU |
| 41 | GUUGGG | 89 | GAUGGG | 137 | UUUGGG | 185 | UAUGGG |
| 42 | GUUGGU | 90 | GAUGGU | 138 | UUUGGU | 186 | UAUGGU |
| 43 | GUUCAG | 91 | GAUCAG | 139 | UUUCAG | 187 | UAUCAG |
| 44 | GUUCAU | 92 | GAUCAU | 140 | UUUCAU | 188 | UAUCAU |
| 45 | GUUCUG | 93 | GAUCUG | 141 | UUUCUG | 189 | UAUCUG |
| 46 | GUUCUU | 94 | GAUCUU | 142 | UUUCUU | 190 | UAUCUU |
| 47 | GUUCGG | 95 | GAUCGG | 143 | UUUCGG | 191 | UAUCGG |
| 48 | GUUCGU | 96 | GAUCGU | 144 | UUUCGU | 192 | UAUCGU |

In another embodiment, present motif removal approach can be carried out on other long polynucleotides of more than 54 nucleotides to decrease the innate immunogenicity of such polynucleotides. In some embodiments, the long polynucleotide comprises at least 54, at least 55, at least 56, at least 57, at least 60, at least 65, at least 70, at least 75, at least 80, at least 85, at least 90, at least 95, at least 100, at least 110, at least 120, at least 130, at least 140, or at least 150 nucleotides. These long polynucleotides include, but are not limited to, guide RNAs (gRNAs) for Crispr-Cas9, long non-coding RNAs (IncRNAs), ribosomal RNAs (rRNAs), transfer RNAs (tRNAs), and circular RNAs (circRNAs).

In some embodiments, a starting, unengineered polynucleotide comprises multiple immunogenic motifs. In some embodiments, the multiple immunogenic motifs in the polynucleotide are motifs of a single type (e.g., every immunogenic motif of the polynucleotide is a motif selected from the group consisting of UCW, UWN, USU, UNU, KWUNDK, KNUWDK, UNUNDK, KNUNDK and KNUNUK, wherein W denotes adenosine monophosphate or uridine monophosphate and S denotes guanosine monophosphate or cytidine monophosphate). In some embodiments, the multiple immunogenic motifs in the polypeptide include different types (e.g., there are at least two different motif types in the polynucleotide sequence selected from the group consisting of UCW, UWN, USU, UNU, KWUNDK, KNUWDK, UNUNDK, KNUNDK and KNUNUK, wherein W denotes adenosine monophosphate or uridine monophosphate and S denotes guanosine monophosphate or cytidine monophosphate).

In some embodiments, precisely sequence engineered polynucleotides display improved functionality, as compared to polynucleotides without the engineering (targeted removal of immunogenic motifs), or as compared to polynucleotides altered in other conventional methods. In some embodiments, the phrase “improved functionality” refers to displaying lower immunogenicity and stealth from innate immune system receptors including, but not limited to, TLR 7 and TLR8). In embodiments where the polynucleotide encodes a protein, the phrase “improved functionality” includes references to improved translational efficiency, which results in improved production and increased amount of the encoded protein. In some embodiments, the phrase “improved functionality” includes references to enhanced stability of the engineered polynucleotide. In a specific embodiment, the enhanced stability of a precise sequence-engineered polynucleotide is due to improved or enhanced resistance to endonucleases and/or exonucleases.

In another embodiment, said motif removal approach may be used in combination with one or more commonly used mRNA chemical modifications, including but not limited to, pseudouridine (ψ), 5-methylcytidine (m5C), N1-methyl-pseudouridine (N1mψ), methoxyuridine (5moU), N6-methyladenosine (m6A), 5-methyluridine (m5U), or 2-thiouridine (s2U), where said modifications replace 0.1-1%, 1-10% or 10-25% or 25-50% or 50-100% of canonical nucleotides in mRNA.

In another embodiment, said motif removal approach may be used in combination with one or more of other naturally found RNA chemical modifications, including but not limited to 1,2′-O-dimethyladenosine, 1,2′-O-dimethylguanosine, 1,2′-O-dimethylinosine, 1-methyl-3-(3-amino-3-carboxypropyl)pseudouridine, 1-methyladenosine, 1-methylguanosine 1-methylinosine, 1-methylpseudouridine, 2,8-dimethyladenosine, 2-methylthiomethylenethio-N6-isopentenyl-adenosine, 2-geranylthiouridine, 2-lysidine, 2-methyladenosine, 2-methylthio cyclic N6-threonylcarbamoyladenosine, 2-methylthio-N6-(cis-hydroxyisopentenyl) adenosine, 2-methylthio-N6-hydroxynorvalylcarbamoyladenosine, 2-methylthio-N6-isopentenyladenosine 2-methylthio-N6-methyladenosine, 2-methylthio-N6-threonylcarbamoyladenosine, 2-selenouridine, 2-thio-2′-O-methyluridine, 2-thiocytidine 2-thiouridine, 2′-O-methyladenosine, 2′-O-methylcytidine, 2′-O-methylguanosine, 2′-O-methylinosine, 2′-O-methylpseudouridine, 2′-O-methyluridine, 2′-O-methyluridine, 5-oxyacetic acid methyl ester, 2′-O-ribosyladenosine (phosphate), 2′-O-ribosylguanosine (phosphate), 3,2′-O-dimethyluridine, 3-(3-amino-3-carboxypropyl)-5,6-dihydrouridine, 3-(3-amino-3-carboxypropyl)pseudouridine, 3-(3-amino-3-carboxypropyl)uridine, 3-methylcytidine, 3-methylpseudouridine, 3-methyluridine, 4-demethylwyosine, 4-thiouridine, 5,2′-O-dimethylcytidine, 5,2′-O-dimethyluridine, 5-(carboxyhydroxymethyl)-2′-O-methyluridine methyl ester, 5-(carboxyhydroxymethyl)uridine methyl ester, 5-(isopentenylaminomethyl)-2-thiouridine, 5-(isopentenylaminomethyl)-2′-O-methyluridine, 5-(isopentenylaminomethyl)uridine, 5-aminomethyl-2-geranylthiouridine, 5-aminomethyl-2-selenouridine, 5-aminomethyl-2-thiouridine, 5-aminomethyluridine, 5-carbamoylhydroxymethyluridine, 5-carbamoylmethyl-2-thiouridine, 5-carbamoylmethyl-2′-O-methyluridine, 5-carbamoylmethyluridine, 5-carboxyhydroxymethyluridine, 5-carboxymethyl-2-thiouridine, 5-carboxymethylaminomethyl-2-geranylthiouridine, 5-carboxymethylaminomethyl-2-selenouridine, 5-carboxymethylaminomethyl-2-thiouridine, 5-carboxymethylaminomethyl-2′-O-methyluridine, 5-carboxymethylaminomethyluridine, 5-carboxymethyluridine, 5-cyanomethyluridine, 5-formyl-2′-O-methylcytidine, 5-formylcytidine, 5-hydroxycytidine, 5-hydroxymethylcytidine, 5-hydroxyuridine, 5-methoxycarbonylmethyl-2-thiouridine, 5-methoxycarbonylmethyl-2′-O-methyluridine, 5-methoxycarbonylmethyluridine, 5-methoxyuridine, 5-methyl-2-thiouridine, 5-methylaminomethyl-2-geranylthiouridine, 5-methylaminomethyl-2-selenouridine, 5-methylaminomethyl-2-thiouridine, 5-methylaminomethyluridine, 5-methylcytidine, 5-methyldihydrouridine, 5-methyluridine, 5-taurinomethyl-2-thiouridine, 5-taurinomethyluridine, 7-aminocarboxypropyl-demethylwyosine, 7-aminocarboxypropylwyosine, 7-aminocarboxypropylwyosine methyl ester, 7-aminomethyl-7-deazaguanosine, 7-cyano-7-deazaguanosine, 7-methylguanosine, 8-methyladenosine, N2,2′-O-dimethylguanosine, N2,7,2′-O-trimethylguanosine, N2,7-dimethylguanosine, N2,N2,2′-O-trimethylguanosine, N2,N2,7-trimethylguanosine, N2,N2-dimethylguanosine, N2-methylguanosine, N4,2′-O-dimethylcytidine, N4,N4,2′-O-trimethylcytidine, N4,N4-dimethylcytidine, N4-acetyl-2′-O-methylcytidine, N4-acetylcytidine, N4-methylcytidine, N6,2′-O-dimethyladenosine, N6,N6,2′-O-trimethyladenosine, N6,N6-dimethyladenosine, N6-(cis-hydroxyisopentenyl)adenosine, N6-acetyladenosine, N6-formyladenosine, N6-glycinylcarbamoyladenosine, N6-hydroxymethyladenosine, N6-hydroxynorvalylcarbamoyladenosine, N6-isopentenyladenosine, N6-methyl-N6-threonylcarbamoyladenosine, N6-methyladenosine, N6-threonylcarbamoyladenosine, Qbase, agmatidine, archaeosine, cyclic N6-threonylcarbamoyladenosine, dihydrouridine epoxyqueuosine, galactosyl-queuosine, glutamyl-queuosine, hydroxy-N6-threonylcarbamoyladenosine, hydroxywybutosine, inosine, isowyosine, mannosyl-queuosine, methylated undermodified hydroxywybutosine, methylwyosine, peroxywybutosine, pseudouridine, queuosine, undermodified hydroxywybutosine, uridine 5-oxyacetic acid, uridine 5-oxyacetic acid methyl ester, wybutosine, and wyosine, where said natural modifications replace 0.1-1%, 1-10% or 10-25% or 25-50% or 50-100% of canonical nucleotides in mRNA.

Messenger RNA immunogenicity and translational activity are also affected by capping, polyadenylation, and impurity (dsRNA contaminant from IVT reaction). In present invention mRNAs were capped enzymatically, using Vaccinia capping system (which caps 5′ end with a 7mG yielding Cap 0 structure, and 2-O-methylates N1-nucleotide yielding Cap I structure at 5′ terminus of mRNA). In another embodiment, sequence engineered mRNAs may be capped co-transcriptionally using a synthetic or natural cap analogue, such as but not limited to, 3″-O-Me-m7G(5′)ppp(5′)G (ARCA) or m7G(5′)ppp(5′)(2′OMeA/G)pG (CleanCap). In another embodiment, mRNAs can be used uncapped, with or without dephosphorylation of the 5′ end (5′ppp).

In some embodiments of the present invention, mRNAs are polyadenylated using a template-based approach. In this approach template DNA sequence contains a terminal polyA/T sequence that encodes a fixed length polyA tail on the mRNA. In an alternative embodiment, sequence engineered mRNAs can be polyadenylated enzymatically using Poly(A) Polymerase. In another embodiment, mRNAs can be used un-polyadenylated.

In some embodiments, mRNAs are purified via reverse phase HPLC followed by size-exclusion chromatography. In another embodiments, mRNAs are purified via ion-exchange chromatography, size exclusion chromatography, affinity chromatography, or enzymatic digestion of dsRNAs with RNAse III or dicer treatment. In another embodiment, a combination of enzymatic digestion and one or more of chromatographic methods may be used.

In present invention, motif removal of mRNA was used without additional sequence engineering methods. However, it is possible to combine this precise mRNA engineering approach with other sequence engineering approaches. In some embodiments, sequence engineering for motif removal are used in combination sequence engineering for codon optimization. In some embodiments, codon optimization is based on codon usage (codon bias), codon neighbor context, mRNA secondary structure, mRNA tertiary structure, or a combination of these parameters. Protein expression yield of mRNA can be significantly improved via codon optimization. This sequence engineering approach can be used together with removal of TLR7 and/or TLR8 sequence motifs.

In another embodiment, precise sequence engineering approach (motif removal) can be combined with a crude sequence engineering approach, such as high GC mRNA, wherein sequence engineering is performed on mRNA to maximize GC content of said mRNA, low GU, wherein sequence engineering is performed to minimize G and U content of mRNA, or low U mRNA, wherein sequence engineering is performed to minimize U content of mRNA. Because these crude approaches usually fall short of complete ablation of immunogenicity, they can be further improved by combining with precise engineering to remove the remaining motifs. In present invention, sequence engineering was performed within coding regions of mRNAs. In another embodiment, 5′ and 3′ untranslated regions can also be engineered to remove immunogenic motifs. In another embodiment, 5′ and 3′ untranslated regions can be selected (from a library of natural or synthetic UTR sequences) to avoid or minimize the number of motifs in these regions.

In some embodiments of the present invention, sequence engineered mRNAs were linear mRNAs. In other embodiments, sequence engineered mRNAs can be circular mRNAs made via chemical, enzymatic, ribozyme-mediated, or self-circularization.

In some embodiments, the present invention employs cationic lipid-based delivery agents. In other embodiments, mRNAs can be delivered by other delivery agents, including but not limited to, polylactide, polylactide-polyglycolide copolymers, polyacrylates, polyalkycyanoacrylates, polycaprolactones, dextran, gelatin, alginate, protamine, collagen, albumin, chitosan, cyclodextrins, PEGylated protamine, poly(L-lysine) (PLL), PEGylated PLL, polyethylenimine (PEI), lipid nanoparticles, liposomes, nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, dry powders, nanodendrimers, starch-based delivery systems, micelles, emulsions, sol-gels, niosomes, plasmids, viruses, virus-like particles, calcium phosphate nucleotides, aptamers, and peptides. In other embodiments, these delivery agents are surface functionalized via conjugation to small molecule ligands, DNA or RNA aptamers, oligopeptides, or proteins such as antibodies, antibody fragments, and ligands such as transferrin.

In present invention, mRNAs were delivered to cells in vitro. In another embodiment, mRNA can be delivered to cells, tissues or organisms ex vivo or in vivo. The delivery route for in vivo administration is oral or parenteral (intravenous, intramuscular, intradermal, or subcutaneous).

In some embodiments, mRNAs encoding a single protein are delivered alone. In another embodiments, multiple mRNAs encoding different proteins are delivered as a cocktail formulation. Individual mRNAs within this formulation may be naked mRNA or may be encapsulated within a lipid nanoparticle or a polymeric carrier allowing reasonable uptake and translation of mRNAs or may be a combination of naked and encapsulated mRNAs. In some embodiments, the cocktail mRNAs are further optimized for activity in specific applications by altering mRNA sequence and/or delivery agent constituents, size, charge, charge ratio, surface chemistry. In specific embodiments, some of the mRNAs in a cocktail formulation are engineered to minimize TLR7/8 binding while others remain un-engineered or partially engineered to allow for selective or partial stimulation of innate immune system.

### EXAMPLES

The following non-limiting examples form part of the present specification and are included to further demonstrate certain aspects of the present disclosure.

### Example 1. Materials and Methods

### Template DNA Generation (IDT)

All DNA templates used in this disclosure included a T7 promoter, a 5′UTR (untranslated region) sequence, a coding region, and a 3′UTR sequence. Coding regions were engineered by altering the wild type eGFP template DNA sequence, where alternative codons encoding the same amino acid residues as the wild type codons were used to either reduced G and U content or remove immunogenic sequence motifs within the open reading frame. Designed sequences were synthesized by a commercial vendor (IDT) and cloned into the pMini-T vector (PCR Cloning Kit, NEB) via TA cloning and sequence verified via Sanger sequencing. Messenger RNA was obtained from the vector by PCR amplification using Q5 High-Fidelity DNA polymerase (NEB) with forward (TTGGACCCTCGTACAGAAGCT) (SEQ ID NO: 5) and reverse (TTTTTTTTTTTTTTTTTTTTTTTTTTTTTTTTTTTTTTTTTTTTTTTTTTTTTTTTTTTTTTTTTTTT TTTTTTTTTTTTTTTTTTTTTTTTTTTTTTTTTTTTTTTTTTTTTTTTTTTTATGGCCAGAAGGC AAGCC) (SEQ ID NO: 6) primers. Reverse primer included the template sequence of a 120 nucleotide-long polyA tail. PCR reaction products were run on an agarose gel and purified with NucleoSpin Gel and PCR Clean-up kit (Macherey-Nagel).

In vitro Transcription (IVT) of mRNAUnmodified mRNAs were transcribed from DNA templates with HiScribe™ T7 High Yield RNA Synthesis Kit using manufacturer's protocols. IVT reaction was run at 37° C. for 2 hours (modified mRNA). Reaction product was treated with TURBO DNase (Thermo Fisher) at 37° C. for 10 minutes and mRNA was isolated with a MEGAclear Transcription Clean-Up kit (Thermo Fisher). Capping was performed post-transcriptionally using Vaccinia Capping System (NEB) and mRNA Cap 2′-O-Methyltransferase (NEB). Phosphatase treatment was carried out with Antarctic Phosphatase enzyme (NEB) followed by isolation with MEGAclear Transcription Clean-Up Kit. Modified eGFP mRNA with pseudouridine and 5-methylcytidine (L-6101) was obtained from TriLink Biotechnologies.

mRNA Purification:

Capped and dephosphorylated mRNA was HPLC purified according to Kariko et al., 2013. Briefly, messenger RNA was run on a Varian Prostar HPLC instrument equipped with a reverse phase PDVB HPLC column (RNASep Column; Concise Seperations) using 0.1 M TEAA (Mobile Phase A) and TEAA with 25% Acetonitrile buffers (Mobile Phase B). Main mRNA fraction was concentrated with an Amicon Ultra-15 centrifugal filter unit (Millipore) and diluted in RNAse-free water. RNA was collected by precipitation in sodium acetate (3M, pH 5.5; Thermo Fisher), isopropanol (Thermo Fisher) and glycogen (Roche), overnight. RNA concentration was measured with NanoDrop 2000 UV-Vis spectrophotometer (Thermo Fisher).

### Cells:

HEK293 TLR8 and its parental line (HEK293 Null) were acquired from Invivogen. Cells were passaged with DMEM (Corning) and 10% FBS (Seradigm). Cell passage numbers at the time of experimentation were less than 15. Human primary monocyte-derived dendritic cells (MDDCs) were obtained from Astarte Biologics (Donor #345). AIM V medium (Thermo Fisher) supplemented with 100 ug/ml GM-CSF and IL-4 (R&D Systems) was used for maintaining MDDCs.

mRNA Transfection:

48 hours before transfection, 20,000-40,000 HEK293 cells were seeded on poly-L-lysine (Sigma) pre-coated 96-well plates. For Lipofectamine 2000 (Thermo Fisher) based transfections, on the day of transfection, medium was replaced with 50 μl of Opti-MEM I serum free medium (Thermo Fisher). For each well, 400 ng of mRNA was mixed with Opti-MEM to final volume of 25 μl and 0.4 μl Lipofectamine 2000 was mixed with 24.6 μl Opti-MEM. Solutions were pre-incubated at room temperature for 5 minutes. They were then combined and incubated at room temperature for 20 minutes. Cells were transfected by adding 50 μl of mRNA-Lipofectamine complexes into each well. Medium was replaced with DMEM and 10% FBS 4 hours after transfection. For repeated (serial) transfection with Lipofectamine 2000, seeded cell number was lowered to 12,000 per well. Cells were seeded on day 0 and transfected on days 2, 3, and 4.

For TransIT mRNA (Mirus Bio) based transfection of HEK293 cells, cells were seeded on poly-L-lysine pretreated 96-well plates at 25,000 cells per well. 72 hours later, 400 ng mRNA, 0.22 μl TransIT mRNA reagent, 0.14 μl TransIT boost reagent, and OptiMEM I serum free medium to a final volume of 17.5 μl was used per well. Medium was replaced with growth medium 24 hours after transfection. For MDDC transfection, frozen cells were thawed, washed and 50,000 cells were plated per well on a 96-well plate. Cells were transfected 24 hours later using 0.11 μl TransIT mRNA and 0.07 μl boost reagent. Medium was replaced 4 hours after transfection.

SEAP and eGFP Quantification

For eGFP quantification, plates were read with EnVision 2105 Multimode Plate Reader. For innate immunogenicity measurements, SEAP activity was measured by QUANTI-Blue Secreted Alkaline Phosphatase Assay (InvivoGen) 22-24 hours after transfection. The incubation for phosphatase assay was performed for 2 hours at 37° C.

### Example 2

In some embodiments, sequence engineering was performed on the ORF (coding region) of template DNAs encoding eGFP mRNAs. Unengineered or native (wild-type) eGFP mRNA with flanking UTR sequences from Tobacco etch virus (5′UTR) and Mus musculus alpha-globin (3′UTR), and a poly-A tail [120 As].

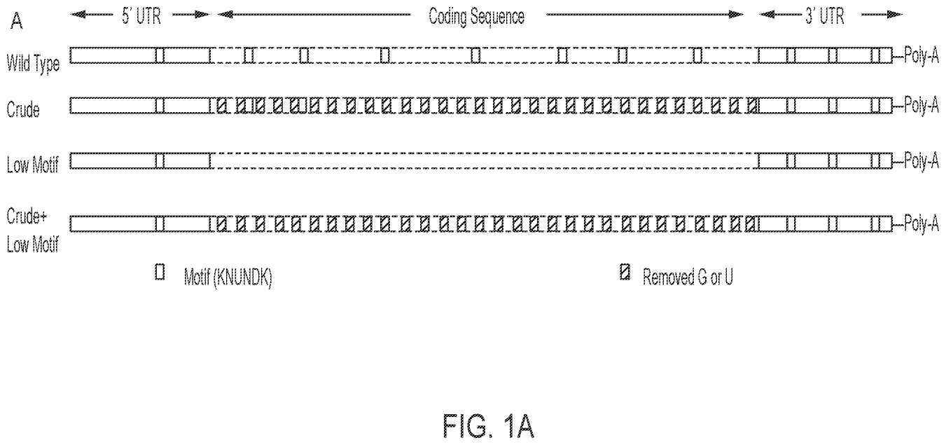

(SEQ ID NO: 1) had 11 immunogenic motifs that are implicated in TLR8 binding, 7 of these were found in the coding region of the mRNA while the remaining 4 were localized within 5′- and 3′UTR regions (FIG. 1A). Crude engineering approach resulted in low GU mRNA (SEQ ID NO: 2), which has 78 total sequence alterations, with 5 of the 7 immunogenic motifs within the coding region being removed. In contrast, precise sequence engineering approach resulted in low motif mRNA (SEQ ID NO: 3) which has very few sequence alterations (7 total) with all of the 7 immunogenic motifs within the coding region being removed (FIG. 1B).

### Example 3

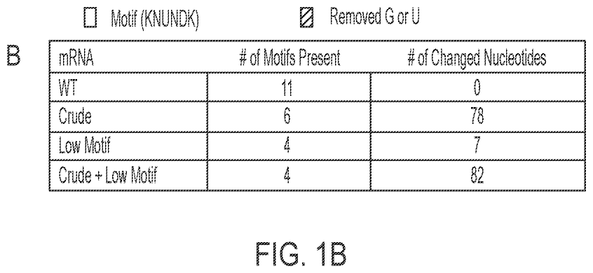

In some embodiments, sequence engineered mRNAs were transfected with Lipofectamine 2000 into HEK293 cells overexpressing TLR8 (FIG. 2). 27,000 cells/well were seeded on a Poly-L-Lysine pretreated 96-well plate. Each well was transfected 48 hours later with 400 ng/well mRNA using Lipofectamine 2000. Medium was replaced after 4 hours. Innate immunogenicity was determined by quantifying SEAP activity in cell culture supernatant 24 hours post transfection (FIG. 2). Reduction of TLR8 stimulation was seen with both low GU mRNA (crude) and low motif mRNA. Combined use of crude and precise approaches (crude+low motif mRNA) did not result in additional reduction in TLR8 activation.

### Example 4

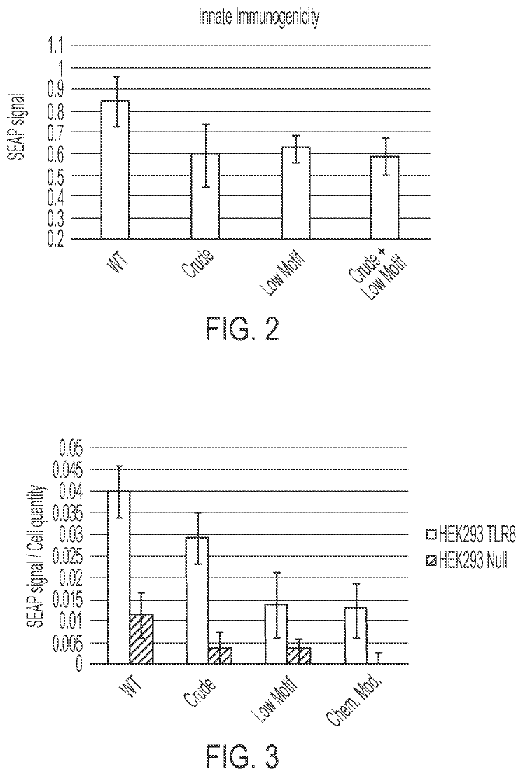

In some embodiments, sequence engineered mRNAs were transfected with TransIT-mRNA reagent into HEK293 cells overexpressing TLR8 or parental HEK293 Null cells without TLR8 overexpression (FIG. 3). 35,000 cells/well were seeded on a Poly-L-Lysine pretreated 96-well plate. Cells were transfected 48 hours later with 400 ng/well of mRNA. Medium was replaced after 4 hours. Innate immunogenicity was determined by quantifying SEAP activity in cell culture supernatant before and 24 hours post transfection. Pre-transfection SEAP reads were used to normalize immune signal to seeded cell quantity. In TransIT-based delivery system, similar to Lipofectamine 2000 based transfection, precise engineering showed reduced TLR8 stimulation. Chemically modified mRNA similarly demonstrated low TLR8 stimulation. While crude approach also showed reduced TLR8 activity, the SEAP signal of low GU mRNA was higher compared to that of low motif mRNA and chemically modified mRNA.

### Example 5

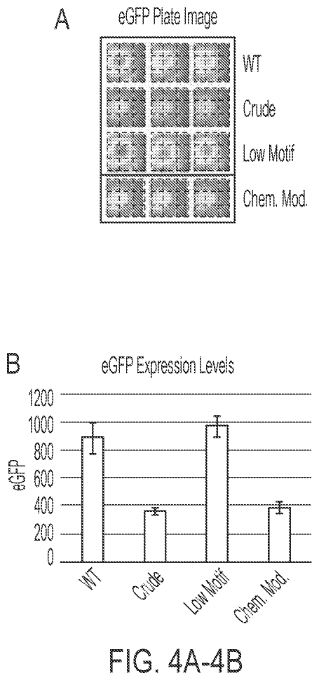

In some embodiments, sequence engineered mRNAs were transfected with Lipofectamine 2000 into HEK293 cells overexpressing TLR8 (FIG. 4). 27,000 cells/well were seeded on a Poly-L-Lysine pretreated 96-well plate. Each well was transfected 48 hours later with 400 ng/well mRNA. Medium was replaced after 4 hours. Protein expression levels of eGFP were determined by imaging the plate (FIG. 4A) and quantifying eGFP signal in each well (FIG. 4B) 6 days post transfection. Based on eGFP expression, crude approach and chemical modification resulted in reduced mRNA translation whereas precise sequence engineering (low motif mRNA) demonstrated preserved translation.

### Example 6

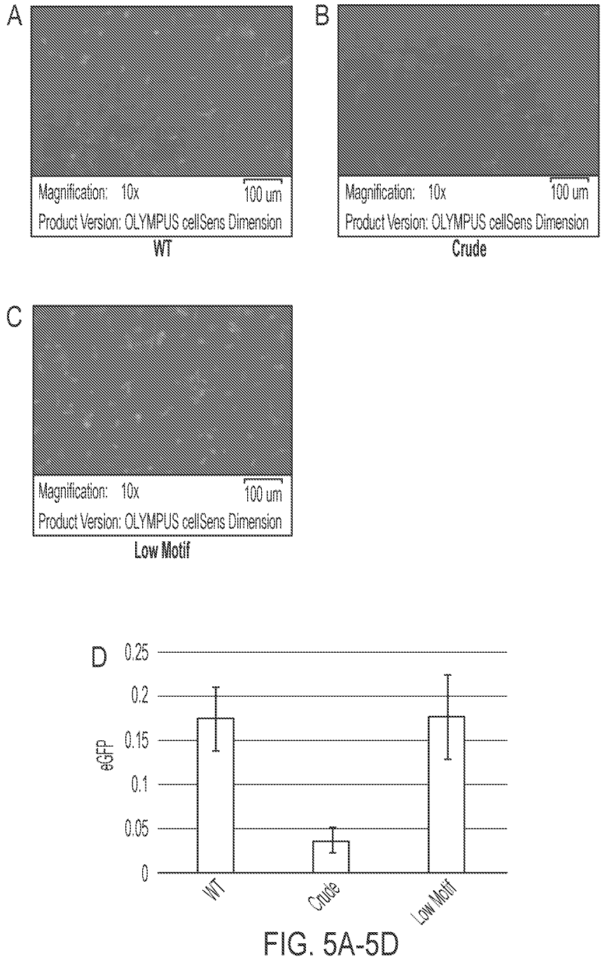

In some embodiments, sequence engineered mRNAs were transfected with TransIT mRNA reagent into MDDCs (FIG. 5). 50,000 cells/well were seeded on a 96-well plate. Each well was transfected 24 hours later with 400 ng/well mRNA. Medium was replaced after 4 hours. Protein expression levels of eGFP were determined by imaging the plate (FIG. 5A) and quantifying eGFP signal in each well (FIG. 5B) 4 days post transfection. Similar to Lipofectamine transfected mRNAs, TransIT transfected mRNA showed improved translational activity of low motif mRNA compared to that of low GU mRNA.

### Example 7

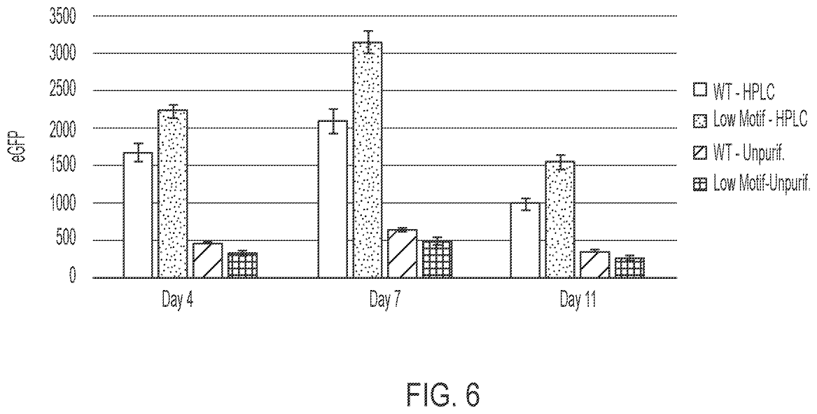

In another specification, sequence engineered mRNAs were transfected repeatedly with Lipofectamine 2000 reagent into HEK293 cells overexpressing TLR8 (FIG. 6). 12,000 cells/well were seeded on Day 0 on a Poly-L-Lysine pretreated 96-well plate. Each well was transfected on Days 2, 3, and 4 with 400 ng/well mRNA. Medium was replaced after each transfection 4 hours. Protein expression levels of eGFP were determined by quantifying eGFP signal in each well (FIG. 5B) on Days 4, 7 and 11. In repeated transfection setting, low motif mRNA showed higher translation than both low GU mRNA and wild-type (unengineered) mRNA.

SEQUENCES

SEQ ID NO: 1. Synthetic Template DNA Sequence for In Vitro Transcription
of Wild type eGFP mRNA. Synthetic DNA sequence comprising T7 phage RNA
Polymerase promoter site, Tobacco etch virus 5′ untranslated region (UTR),
Native (Wild type) version of Aequorea victoria enhanced green fluorescent
protein (eGFP) coding sequence, mus musculus alpha-globin 3′UTR, and poly-A
tail [120 As].
TTGGACCCTCGTACAGAAGCTAATACGACTCACTATAGGGAAATAAGAGAGAAAAG
AAGAGTAAGAAGAAATATAAGAGCCACCATGGTGAGCAAGGGCGAGGAGCTGTTCA
CCGGGGTGGTGCCCATCCTGGTCGAGCTGGACGGCGACGTAAACGGCCACAAGTTC
AGCGTGTCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCACCCTGAC
CTACGGCGTGCAGTGCTTCAGCCGCTACCCCGACCACATGAAGCAGCACGACTTCTT
CAAGTCCGCCATGCCCGAAGGCTACGTCCAGGAGCGCACCATCTTCTTCAAGGACGA
CGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAG
CTGGAGTACAACTACAACAGCCACAACGTCTATATCATGGCCGACAAGCAGAAGAAC
GGCATCAAGGTGAACTTCAAGATCCGCCACAACATCGAGGACGGCAGCGTGCAGCT
CGCCGACCACTACCAGCAGAACACCCCCATCGGCGACGGCCCCGTGCTGCTGCCCG
ACAACCACTACCTGAGCACCCAGTCCGCCCTGAGCAAAGACCCCAACGAGAAGCGC
GATCACATGGTCCTGCTGGAGTTCGTGACCGCCGCCGGGATCACTCTCGGCATGGAC
GAGCTGTACAAGTAAGCTGCCTTCTGCGGGGCTTGCCTTCTGGCCATGCCCTTCTTCTC
TCCCTTGCACCTGTACCTCTTGGTCTTTGAATAAAGCCTGAGTAGGAAGAAAAAAAAA
AAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAA
A
SEQ ID NO: 2. Synthetic Template DNA Sequence for In Vitro Transcription of
Crude Engineered (Low GU) eGFP mRNA, Coding Sequence: G- and U-reduced
Aequorea victoria eGFP coding sequence.
ATGGTCAGCAAAGGCGAAGAACTCTTCACCGGCGTCGTCCCCATCCTCGTCGAACTC
GACGGCGACGTAAACGGCCACAAGTTCAGCGTCTCCGGCGAAGGCGAAGGCGACGC
CACCTACGGCAAACTCACCCTCAAATTCATCTGCACCACCGGCAAACTCCCCGTCCCC
TGGCCCACCCTCGTCACCACCTTCACCTACGGCGTCCAATGCTTCAGCCGCTACCCCG
ACCACATGAAACAACACGACTTCTTCAAAAGCGCCATGCCCGAAGGCTACGTCCAAG
AACGCACCATCTTCTTCAAAGACGACGGCAACTACAAAACCCGCGCCGAAGTCAAAT
TCGAAGGCGACACCCTCGTCAACCGCATCGAACTCAAAGGCATCGACTTCAAAGAAG
ACGGCAACATCCTAGGCCACAAACTCGAATACAACTACAACAGCCACAACGTCTACA
TCATGGCCGACAAACAAAAAAACGGCATCAAAGTCAACTTCAAAATCCGCCACAACA
TCGAAGACGGCAGCGTCCAACTCGCCGACCACTACCAACAAAACACCCCCATCGGCG
ACGGCCCCGTCCTCCTCCCCGACAACCACTACCTCAGCACCCAATCCGCCCTAAGCA
AAGACCCCAACGAAAAACGCGACCACATGGTCCTCCTCGAATTCGTCACCGCCGCCG
GCATCACCCACGGCATGGACGAACTCTACAAATAA
SEQ ID NO: 3. Synthetic Template DNA Sequence for In Vitro Transcription of
Low Motif eGFP mRNA. Coding Sequence: KNUNDK motif removed Aequorea
victoria eGFP coding sequence.
ATGGTCAGCAAAGGCGAAGAACTCTTCACCGGCGTCGTCCCCATCCTCGTCGAACTC
GACGGCGACGTAAACGGCCACAAGTTCAGCGTCTCCGGCGAAGGCGAAGGCGACGC
CACCTACGGCAAACTCACCCTCAAATTCATCTGCACCACCGGCAAACTCCCCGTCCCC
TGGCCCACCCTCGTCACCACCTTCACCTACGGCGTCCAATGCTTCAGCCGCTACCCCG
ACCACATGAAACAACACGACTTCTTCAAAAGCGCCATGCCCGAAGGCTACGTCCAAG
AACGCACCATCTTCTTCAAAGACGACGGCAACTACAAAACCCGCGCCGAAGTCAAAT
TCGAAGGCGACACCCTCGTCAACCGCATCGAACTCAAAGGCATCGACTTCAAAGAAG
ACGGCAACATCCTAGGCCACAAACTCGAATACAACTACAACAGCCACAACGTCTACA
TCATGGCCGACAAACAAAAAAACGGCATCAAAGTCAACTTCAAAATCCGCCACAACA
TCGAAGACGGCAGCGTCCAACTCGCCGACCACTACCAACAAAACACCCCCATCGGCG
ACGGCCCCGTCCTCCTCCCCGACAACCACTACCTCAGCACCCAATCCGCCCTAAGCA
AAGACCCCAACGAAAAACGCGACCACATGGTCCTCCTCGAATTCGTCACCGCCGCCG
GCATCACCCACGGCATGGACGAACTCTACAAATAA
SEQ ID NO: 4. Synthetic Template DNA Sequence for In Vitro Transcription of
Crude (Low GU) and Low Motif eGFP mRNA. Coding Sequence: KNUNDK motif
removed and GU reduced Aequorea victoria eGFP.
ATGGTCAGCAAAGGCGAAGAACTCTTCACCGGCGTCGTCCCCATCCTCGTCGAACTC
GACGGCGACGTAAACGGCCACAAGTTCAGCGTCTCCGGCGAAGGCGAAGGCGACGC
CACCTACGGCAAACTCACCCTCAAATTCATCTGCACCACCGGCAAACTCCCCGTCCCC
TGGCCCACCCTCGTCACCACCTTCACCTACGGCGTCCAATGCTTCAGCCGCTACCCCG
ACCACATGAAACAACACGACTTCTTCAAAAGCGCCATGCCCGAAGGCTACGTCCAAG
AACGCACCATCTTCTTCAAAGACGACGGCAACTACAAAACCCGCGCCGAAGTCAAAT
TCGAAGGCGACACCCTCGTCAACCGCATCGAACTCAAAGGCATCGACTTCAAAGAAG
ACGGCAACATCCTAGGCCACAAACTCGAATACAACTACAACAGCCACAACGTCTACA
TCATGGCCGACAAACAAAAAAACGGCATCAAAGTCAACTTCAAAATCCGCCACAACA
TCGAAGACGGCAGCGTCCAACTCGCCGACCACTACCAACAAAACACCCCCATCGGCG
ACGGCCCCGTCCTCCTCCCCGACAACCACTACCTCAGCACCCAATCCGCCCTAAGCA
AAGACCCCAACGAAAAACGCGACCACATGGTCCTCCTCGAATTCGTCACCGCCGCCG
GCATCACCCACGGCATGGACGAACTCTACAAATAA
SEQ ID NO: 5. DNA-Artificial sequence-Oligonucleotide
TTGGACCCTCGTACAGAAGCT
SEQ ID NO: 6. DNA-Artificial sequence-Oligonucleotide
TTTTTTTTTTTTTTTTTTTTTTTTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTTTTTATGGCCAGAAGGC
AAGCC

What is claimed is:

1. An engineered polynucleotide whose sequence corresponds to that of a reference oligonucleotide that encodes a polypeptide and includes a plurality of TLR7 motifs or TLR8 motifs within its polypeptide-coding sequences, except that the engineered polynucleotide lacks each of the motifs of the plurality but still encodes the polypeptide.

2. The engineered polynucleotide of claim 1, wherein each of the motifs is selected from the group consisting of KNUNDK motifs UCW motifs, UNU motifs, UWN motifs, USU motifs, KWUNDK motifs, KNUWDK motifs, UNUNDK motifs, KNUNUK motifs, and combinations thereof.

3. The engineered polynucleotide of claim 1 or claim 2, which is or comprises DNA.

4. The engineered polynucleotide of claim 1 or claim 2, which is or comprises RNA.

5. A method comprising administering an engineered polynucleotide of claim 1 to a cell.

6. The method of claim 5, wherein the engineered polynucleotide is or comprises RNA.

7. The method of claim 6, wherein the RNA was expressed from a DNA that is also an engineered polynucleotide of claim 1.

8. A method of producing a therapeutic mRNA by expressing it from an engineered DNA whose sequence corresponds to that of a reference DNA that encodes a polypeptide and includes a plurality of TLR7 motifs or TLR8 motifs within its polypeptide-coding sequences, except that the engineered DNA lacks each of the motifs of the plurality but still encodes the polypeptide.

9. An engineered polynucleotide comprising at least 54 nucleotides, wherein the engineered polynucleotide is precisely sequence engineered based on a starting polynucleotide to remove at least one immunogenic sequence motif in the starting polynucleotide.

10. The engineered polynucleotide of claim 9, wherein the starting polynucleotide is a naturally occurring polynucleotide.

11. The engineered polynucleotide of claim 9, wherein the polynucleotide is a synthetic polynucleotide.

12. The engineered polynucleotide according to any one of claims 9-11, wherein the starting polynucleotide is a messenger RNA (mRNA).

13. The engineered polynucleotide of claim 10, wherein the at least one immunogenic sequence motif is removed from at least one region of the mRNA selected from the coding region, the 3′ untranslated region (3′UTR), or the 5′ untranslated region (5′UTR).

14. The engineered polynucleotide of claim 12, wherein the mRNA encodes a polypeptide selected from the group consisting of mammalian proteins, pathogenic antigens, cancer antigens and neoantigens, chimeric proteins, mutated proteins, and synthetic proteins.

15. The engineered polynucleotide of claim 13, wherein the protein encoded by the engineered mRNA has the same amino acid sequence as that of the protein encoded by the starting mRNA sequence.

16. The engineered polynucleotide of claim 9, wherein the engineered polynucleotide is a guide RNA (g RNA) for Crispr-Cas9, long non-coding RNA (IncRNA), tRNA, ribosomal RNA (rRNAs), circular RNA, aptamer RNA, synthetic RNA.

17. The engineered polynucleotide of claim 9, wherein the immunogenic sequence motif comprises a sequence or a plurality of sequences that can bind human TLR7.

18. The engineered polynucleotide of claim 9, wherein the at least one immunogenic sequence motif comprises a sequence or a plurality of sequences that can bind human TLR8.

19. The engineered polynucleotide of claim 18, wherein the immunogenic motif is KNUNDK, wherein K denotes guanosine monophosphate or uridine monophosphate, N denotes any nucleotide, U denotes uridine monophosphate, and D denotes adenosine monophosphate, guanosine monophosphate, or uridine monophosphate.

20. The engineered polynucleotide of claim 9, wherein the immunogenic motif is a motif selected from the group consisting of UCW, UWN, USU, UNU, KWUNDK, KNUWDK, UNUNDK, and KNUNUK, wherein W denotes adenosine monophosphate or uridine monophosphate and S denotes guanosine monophosphate or cytidine monophosphate.

21. The engineered polynucleotide of claim 9, wherein at least 1%, at least 50%, or at least 90% of the immunogenic motif sequences found in the starting polynucleotide sequence are removed.

22. The engineered polynucleotide of claim 9, wherein the precise sequence engineering via immunogenic motif removal is used in combination with codon optimization of the polynucleotide.

23. The engineered polynucleotide of claim 9, wherein the precise sequence engineering is used in combination with at least one of the crude sequence engineering methods selected from the group consisting of low GU content, low U content, and increased GC content-based mRNA sequence engineering.

24. The engineered polynucleotide of claim 9, wherein the precise sequence engineering is used in combination with at least partial chemical modification of the polynucleotide using at least one non-canonical nucleotide selected from the group consisting of pseudouridine (ψ), 5-methylcytidine (m5C), N1-methyl-pseudouridine (N1mψ), 5-methoxyuridine (5moU), N6-methyladenosine (m6A), 5-methyluridine (m5U), or 2-thiouridine (s2U).

25. The engineered polynucleotide of claim 9, wherein the engineered polynucleotide further comprises a 5′cap structure added via enzymatic capping or co-transcriptional capping using a cap analogue.

26. The engineered polynucleotide of claim 9, wherein the engineered polynucleotide further comprises a poly-A tail.

27. The engineered polynucleotide of claim 9, wherein the engineered polynucleotide is purified.

28. A pharmaceutical composition comprising the engineered polynucleotide of claim 1.

29. A veterinary composition or a research-use composition comprising the engineered polynucleotide of claim 9.

30. A delivery vehicle comprising the engineered polynucleotide of claim 9, wherein the delivery vehicle is selected from a group consisting of ionizable or cationic lipid nanoparticles, liposomes, lipoplexes, and polymeric carriers.

31. A method of precise sequence engineering comprising
a) providing a polynucleotide that comprises at least 54 nucleotides;
b) identifying at least one immunogenic motif in the polynucleotide sequence;
c) removing the identified at least one immunogenic motif sequence.

32. The method of claim 31, wherein the polynucleotide is a naturally occurring polynucleotide.

33. The method of claim 31, wherein the polynucleotide is a synthetic polynucleotide.

34. The method according to any one of claims 31-33, wherein the polynucleotide is a messenger RNA (mRNA).

35. The method of claim 34, wherein the modification does not alter the amino acid sequence encoded by the mRNA.

36. The method of any one of claims 31-35, wherein the at least one immunogenic motif identified in step (b) comprises a plurality of immunogenic motifs.

37. The method of claim 36, wherein step c) comprises removing multiple identified immunogenic motifs.

38. The method of claim 36, wherein step (c) comprises removing at least 10% of the identified immunogenic motifs.

39. The method of claim 36, wherein step (c) comprises removing at least 50% of the identified immunogenic motifs.

40. The method of claim 36, wherein step (c) comprises removing all of the identified immunogenic motifs.

41. The method according to any one of claims 31-33, wherein the polynucleotide is selected from the group consisting of a guide RNA (g RNA) for Crispr-Cas9, long non-coding RNA (IncRNA), tRNA, ribosomal RNA (rRNAs), circular RNA, aptamer RNA, and a synthetic RNA.

42. The method of claim 31 further comprising
d) codon optimizing the polynucleotide sequence.

43. The method of claim 31 further comprising
d) performing partial chemical modification of the polynucleotide using at least one non-canonical nucleotide selected from the group consisting of pseudouridine (ψ), 5-methylcytidine (m5C), N1-methyl-pseudouridine (N1mψ), 5-methoxyuridine (5moU), N6-methyladenosine (m6A), 5-methyluridine (m5U), or 2-thiouridine (s2U).

44. The method of claim 31 further comprising adding to the polynucleotide a 5′cap structure via enzymatic capping or co-transcriptional capping using a cap analogue.

45. The method of claim 31 further comprising adding to the polynucleotide a poly-A tail.

46. The method of claim 31 further comprising purifying the polynucleotide.


SEQUENCE LISTING (6 sequences)

SEQ ID NO: 1 - DNA, 1017 bp, Artificial Sequence
  Oligonucleotide
ttggaccctcgtacagaagctaatacgactcactatagggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 720 bp, Artificial Sequence
  Oligonucleotide
atggtcagcaaaggcgaagaactcttcaccggcgtcgtccccatcctcgtcgaactcgacggcgacgtaaacggccacaagttcagcgtctccggcgaaggcgaaggcgacgccacctacggcaaactcaccctcaaattcatctgcaccaccggcaaactccccgtcccctggcccaccctcgtcaccaccttcacctacggcgtccaatgcttcagccgctaccccgaccacatgaaacaacacgacttcttcaaaagcgccatgcccgaaggctacgtccaagaacgcaccatcttcttcaaagacgacggcaactacaaaacccgcgccgaagtcaaattcgaaggcgacaccctcgtcaaccgcatcgaactcaaaggcatcgacttcaaagaagacggcaacatcctaggccacaaactcgaatacaactacaacagccacaacgtctacatcatggccgacaaacaaaaaaacggcatcaaagtcaacttcaaaatccgccacaacatcgaagacggcagcgtccaactcgccgaccactaccaacaaaacacccccatcggcgacggccccgtcctcctccccgacaaccactacctcagcacccaatccgccctaagcaaagaccccaacgaaaaacgcgaccacatggtcctcctcgaattcgtcaccgccgccggcatcacccacggcatggacgaactctacaaataa

SEQ ID NO: 3 - DNA, 720 bp, Artificial Sequence; identical to SEQ ID NO: 2
  Oligonucleotide

SEQ ID NO: 4 - DNA, 720 bp, Artificial Sequence; identical to SEQ ID NO: 2
  Oligonucleotide

SEQ ID NO: 5 - DNA, 21 bp, Artificial Sequence
  Oligonucleotide
ttggaccctcgtacagaagct

SEQ ID NO: 6 - DNA, 138 bp, Artificial Sequence
  Oligonucleotide
ttttttttttttttttttttttttttttttttttttttttttttttttttttttttttttttttttttttttttttttttttttttttttttttttttttttttttttttttttttttttatggccagaaggcaagcc